(19) United States
(12) Patent Application Publication
(10) Pub. No.: US 2005/0002887 A1
(43) Pub. Date: Jan. 6, 2005

(54) Cosmetic composition comprising at least one branched sulphonic polyester and at least one conditioning agent

(75) Inventors: Isabelle Rollat-Corvol, Boulogne (FR); Henri Samain, Bievres (FR)

Correspondence Address: FINNEGAN, HENDERSON, FARABOW, GARRETT & DUNNER;LLP, 1300 I STREET, NW, WASHINGTON, DC, 20005, US

(73) Assignee: L'Oreal S.A.

(21) Appl. No.: 10/706,048

(22) Filed: Nov. 13, 2003

Related U.S. Application Data
(63) Continuation of application No. 09/330,075, filed on Jun. 11, 1999, now abandoned.

(30) Foreign Application Priority Data
Jun. 11, 1998 (FR) 98 07375

(51) Int. Cl. A61K 7/06; A61K 7/11

(52) U.S. Cl. 424/70.11

(57) ABSTRACT

The invention relates to a cosmetic composition for keratin fibers such as the hair, comprising at least one branched sulphonic polyester and at least one conditioning agent chosen from the group comprising non-volatile silicones, cationic or amphoteric polymers and cationic or amphiphilic surfactants. The invention is also directed towards a process for treating keratin fibers such as the hair, in particular a process for fixing and/or holding the hairstyle using the said composition, as well as to the use of this composition in or for the manufacture of a cosmetic hair-styling formulation.

The invention relates to a cosmetic composition for keratin fibers such as the hair, comprising at least one branched sulphonic polyester and at least one conditioning agent chosen from the group comprising non-volatile silicones, cationic and amphoteric polymers and cationic and amphiphilic surfactants. The invention is also directed towards a process for treating keratin fibers such as the hair, in particular a process for fixing and/or holding the hairstyle using the inventive composition, as well as to the use of this composition in or for the manufacture of a cosmetic hair-styling formulation.

For the purposes of the present invention, the term “keratin fibers” means the hair, the eyelashes and the eyebrows.

Fixing of the hairstyle is an important element of styling which involves holding the shape already produced or shaping the hair and simultaneously fixing it.

The hair products for shaping and/or holding the hairstyle which are the most widely available on the cosmetics market are spray compositions essentially containing a solution, usually an alcoholic or aqueous solution, and one or more materials, generally polymer resins, whose function is to form welds between the hair, these also being known as fixing materials, as a mixture with various cosmetic adjuvants. This solution is generally packaged either in a suitable aerosol container placed under pressure with the aid of a propellant, or in a pump-dispenser bottle.

Styling gels or mousses which are generally applied to wet hair before blow-drying or setting are also known. Unlike conventional aerosol lacquers, these compositions have the drawback of not allowing the hair to b fixed in a shape which has already been produced. The reason for this is that these compositions are essentially aqueous and, when applied, they wet the hair and thus cannot maintain the initial shape of the hairstyle. In order to shape and fix the hairstyle, it is thus necessary to subsequently carry out a blow-drying or drying operation.

Most of the compositions of the prior art have the same drawback of not easily allowing the user to restyle the hair to restore its initial shape when the hairstyle has been undone under the effect of a constraint. In this case, it is often necessary to recommence all of the styling and fixing operations if it is desired to regain the initial hairstyle.

Styling compositions are thus sought which allow the shape of the hairstyle to be restored easily when it has been undone, for example by a gust of wind or by vigorous shaking. This capacity is referred to as the “restyling” power, i.e. the possibility of easily restoring the shape of a hairstyle with the minimum effort, without the aid of a comb or a brush, when this hairstyle has been undone.

Another drawback of the compositions of the prior art is that they do not always allow all the hair to be suitably managed, i.e. to make all the hairs take the same shape and the same direction. This occasionally results in an impression of disorder of the hairstyle and an unmanaged appearance. Compositions which afford a manageability effect which is great enough for the head of hair to have a well-groomed appearance are thus sought.

Lastly, compositions intended for fixing the hairstyle occasionally have the drawback of damaging the cosmetic properties of the hair. Thus, the hair can become coarse, difficult to disentangle, lose its pleasant feel and appearance or lack body. Styling compositions which afford good cosmetic properties, in particular in terms of disentangling, softness, feel and body, are thus sought. For the purposes of the present invention, a head of hair will be said to have “body” if it gives a sensation of fullness and of light rigidity when touched by hand.

There is thus a need to find cosmetic compositions, in particular for styling, which do not have all the drawbacks mentioned above.

The inventors have discovered, surprisingly and unexpectedly, that when branched sulphonic polyesters are combined with conditioning agents, it is possible to obtain cosmetic compositions which satisfy the requirements expressed above.

A subject of the invention is thus a cosmetic composition comprising, in a cosmetically acceptable medium, at least one branched sulphonic polyester and at least one conditioning agent chosen from the group comprising non-volatile silicones, cationic and amphoteric polymers and cationic and amphiphilic surfactants.

The branched sulphonic polyesters towards which the present invention is particularly directed are those described in patent applications WO 95/18191, WO 97/08261 and WO 97/20899, the disclosures of which are specifically incorporated by reference herein.

The branched sulphonic polyesters are advantageously formed by polymerization of:

(i) at least one difunctional dicarboxylic acid bearing no sulphonic functions;

(ii) at least one difunctional monomer bearing at least one sulphonic function, the functional group(s) being chosen from the group comprising hydroxyl, carboxyl and amino groups;

(iii) at least one diol or a mixture of at least one diol and at least one diamine;

(iv) optionally one difunctional monomer chosen from the group comprising hydroxycarboxylic acids, aminocarboxylic acids and mixtures thereof;

(v) at least one multifunctional reagent bearing at least three functional groups chosen from the group comprising amino, alcohol and carboxylic acid groups.

This polymerization is advantageously carried out using:

(i) at least one difunctional dicarboxylic acid bearing no sulphonic functions;

(ii) 2 to 15 mol % of difunctional monomer bearing at least one sulphonic function;

(iii) at least one diol or a mixture of at least one diol and at least one diamine;

(iv) 0 to 40 mol % of the difunctional monomer chosen from the group comprising hydroxycarboxylic acids, aminocarboxylic acids and mixtures thereof;

(v) 0.1 to 40 mol % of the multifunctional reagent bearing at least three reactive functional groups.

The branched sulphonic polymers advantageously contain substantially equal proportions, in terms of number of equivalents, of carboxylic acid functions, on the one hand, and of diol and/or diol and diamine functions, on the other hand.

Preferably, the polymers AQ 1350, AQ 1045, AQ 1950 or AQ 14,000 sold by the Eastman Chemical Company are chosen as branched sulphonic polymers, and more preferably the polymer AQ 1350.

The difunctional dicarboxylic acid (i) may advantageously be chosen from the group comprising aliphatic dicarboxylic acids, alicyclic dicarboxylic acids, aromatic dicarboxylic acids and a mixture thereof. More particularly, the dicarboxylic acid (i) may be chosen from the group comprising 1,4-cyclohexanedioic acid, succinic acid, glutaric acid, adipic acid, azelaic acid, sebacic acid, fumaric acid, maleic acid, 1,3-cyclohexanedioic acid, phthalic acid, terephthalic acid and isophthalic acid.

The difunctional monomer (ii) as defined above is advantageously chosen from the group comprising dicarboxylic acids, dicarboxylic acid esters, glycols and hydroxy acids each containing at least one metal sulphonate group.

The diol (iii) is preferably chosen from the group comprising alkanediols and polyalkylenediols, and more particularly this diol (iii) is chosen from the group comprising ethylene glycol, propylene glycol, diethylene glycol, triethylene glycol and polypropylene glycol.

The diamine (iii) can advantageously be chosen from the group comprising alkanediamines and polyalkylenediamines, and the multifunctional reagent (v) is preferably chosen from the group comprising trimethylolethane, trimethylolpropane, glycerol, pentaerythritol, sorbitol, trimellitic anhydride, erythritol, threitol, dipentaerythritol, pyromellitic dianhydride and dimethylpropionic acid.

The composition advantageously comprises, as a relative percentage by weight of the composition, from 0.01 to 40% of branched sulphonic polyester, preferably from 0.1 to 16% of branched sulphonic polyester, and more preferably from 0.5 to 8% of branched sulphonic polyester.

The composition advantageously comprises, as a relative percentage by weight of the composition, from 0.01 to 16% of conditioning agent and preferably from 0.05 to 4%.

The branched sulphonic polyester(s) can be in dissolved form or in the form of a dispersion of solid particles.

A conditioning agent which is insoluble in the group comprising poly-α-olefins, fluoro oils, plant oils, natural waxes, fluoro waxes, fluoro gums, fatty acid esters, insoluble silicones and amide compounds comprising at least one fatty chain may advantageously be chosen; it being possible for the conditioning agent to be present in the form of mixtures.

When the conditioning agent is chosen from cationic and amphoteric polymers, it is advantageously chosen from the group formed by:
(a) cationic cellulose derivatives;
(b) dimethyldiallylammonium halide homopolymers and copolymers;
(c) methacryloyloxyethyltrimethylammonium halide homopolymers and copolymers;
(d) polyquaternary ammonium polymers;
(e) vinylpyrrolidone copolymers containing cationic units;
(f) cationic polysiloxanes.

The cationic polymer is advantageously chosen from quaternary cellulose ether derivatives, copolymers of cellulose with a water-soluble quaternary ammonium monomer, cyclopolymers, cationic polysaccharides, cationic silicone polymers, quaternized and non-quaternized vinylpyrrolidone/dialkylaminoalkyl acrylate and methacrylate copolymers, quaternary polymers of vinylpyrrolidone and of vinylimidazole, polyamidoamines and mixtures thereof.

Water-insoluble cationic surfactants from the group comprising fatty amines and salts thereof, and quaternary ammonium salts, can advantageously be chosen. Preferably, the fatty amines are chosen from the group comprising dioctylamine, stearyldimethylamine, palmityldimethylamine, oleocetyldimethylamine and amidoamines such as stearylamidoethyldiethylamine, behenylamidopropyldimethylamine, stearylamidopropyldimethylamine, oleylamidopropyldimethylamine and stearylamidoethyldimethylamine.

The cationic surfactant of quaternary ammonium salt type is advantageously chosen from those which have the general formula (I) below:

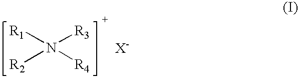

in which R₁ to R₄, which may be identical or different, are chosen from aliphatic radicals comprising from 1 to 22 carbon atoms and aromatic, alkoxy, polyoxyalkylene, alkylamide, hydroxyalkyl, aryl and alkylaryl radicals comprising from 12 to 22 carbon atoms; X is an anion chosen from the group comprising halides, phosphates, acetates, lactates and alkylsulphates,

or a mixture of quaternary ammonium salts of imidazolinium, of formula (II) below:

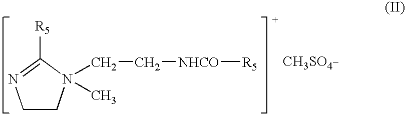

in which R₅ is chosen from alkenyl and alkyl radicals comprising from 13 to 21 carbon atoms and fatty acid derivatives of tallow,

or the quaternary diammonium salts of formula (III):

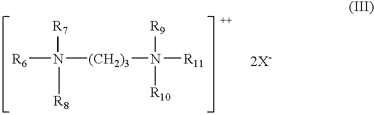

in which R₆ is chosen from aliphatic radicals comprising from 16 to 22 carbon atoms, R₇, R₈, R₉, R₁₀ and R₁₁ are chosen from hydrogen and alkyl radicals comprising from 1 to 4 carbon atoms, and X is an anion chosen from halides, acetates, phosphates and sulphates.

The composition also advantageously comprises common cosmetic additives such as plasticizers or neutralizing agents and it is preferably in the form of a vaporizable composition, a mousse, a gel or a lotion.

The composition preferably comprises, in addition, a solvent chosen from water, an alcohol or an aqueous-alcoholic mixture, as well as a suitable amount of a propellant advantageously chosen from the usual compressed and liquefied gases, preferably compressed air, carbon dioxide or nitrogen, or alternatively a gas which is soluble or insoluble in the composition, such as dimethyl ether, fluoro or non-fluoro hydrocarbons, and mixtures thereof.

Another subject of the invention is an aerosol device comprising a container containing an aerosol composition comprising, on the one hand, a liquid phase (or fluid) containing a composition in accordance with the invention, in a suitable solvent and a propellant, as well as a means for dispensing the said aerosol composition.

Yet another subject of the invention is a process for treating keratin fibers, in particular the hair, which comprises the application of the composition in accordance with the invention to the said fibers, before or after shaping the hairstyle.

Yet another subject of the invention relates to the use of a composition defined above in or for the manufacture of a cosmetic styling formulation.

The examples below illustrate the present invention without limiting its scope.

The polymers indicated below are used.

| | |
|---|---|
| AQ 1350, AQ 1045, AQ 1950 | Branched sulphonic polyesters sold by |
| and AQ 14,000 | Eastman |
| DC 190 | Oxyethylenated and oxypropylenated |
| | polydimethylmethylsiloxane sold by Dow |
| | Corning |
| DC 939 | Cationic silicone sold by Dow Corning |

### EXAMPLE 1

The performance characteristics obtained with compositions in accordance with the invention and with compositions in accordance with the prior art, containing no conditioning agent, were compared. The performance characteristics compared were the disentangling, the body, the softness, the feel, the styling power and the restyling power.

Composition 1 (invention):

| | |
|---|---|
| AQ 1350 | 4 g |
| Dow Corning 190 Surfactant | 0.4 g (active material) |
| Water qs | 100 g |

Composition 2 (invention):

| | |
|---|---|
| AQ 1350 | 4 g |
| DC 939 | 0.4 g |
| Water qs | 100 g |

Composition 3 (prior art)

| | |
|---|---|
| AQ 1350 | 4 g |
| Water qs | 100 g |

Flexible heads were provided for this test. The compositions were applied in a half-head manner, i.e. to the right or left side of the head of hair, separated by a strip in the middle.

3 grams of product were applied per half-head, the hair on which had been shampooed beforehand and then drained dry. The flexible heads were blow-dried, styled at 21° C. and at 30% relative humidity and were left to stand for 10 minutes under these same temperature and humidity conditions.

The performance characteristics obtained were measured by means of a sensory test. The performance characteristics were graded between 0 (poor) and 5 (excellent). They are collated in Table 1 below.

TABLE 1

| | | Composition 2/ |
|---|---|---|
| Comparative [symbol] | Composition 1/Composition 3 | Composition 3 |
| DISENTANGLING | 4.5/3.5 | 5.0/2.0 |
| BODY | 1.5/0.5 | 1.5/0.5 |
| SOFTNESS | 4.0/2.5 | 3.5/1.5 |
| FEEL | 4.0/2.0 | 4.0/1.0 |
| STYLING POWER | 4.0/2.0 | 4.5/3.5 |
| RESTYLING | 4.0/0.0 | 4.0/1.0 |
| POWER | | |

It was observed that the performance characteristics obtained with inventive compositions 1 and 2 were markedly better than those obtained with composition 3. In particular, the restyling power was markedly better, as was the body of the hairstyle.

### EXAMPLE 2

Three compositions in accordance with the invention were prepared.

Composition 5 (invention):

| | |
|---|---|
| AQ 1045 | 1.2 g |
| Dow Corning 190 Surfactant | 0.2 g (active material) |
| Water qs | 100 g |

Composition 6 (invention):

| | |
|---|---|
| AQ 1950 | 8.3 g |
| DC 939 | 1 g |
| Water qs | 100 g |

Composition 7 (invention):

| | |
|---|---|
| AQ 14,000 | 10 g |
| DC 939 | 1 g |
| Water qs | 100 g |

The three compositions 5, 6 and 7 were applied to sensitized (bleached) hair and the hair was left to dry and then brushed.

It was observed that the inventive compositions afforded a substantial softening effect and that the hair was particularly shiny and easy to disentangle.

1. A cosmetic composition for treating human keratin fibers comprising:
at least one branched sulphonic polyester; and
at least one conditioning agent chosen from non-volatile silicones, cationic and amphoteric polymers and cationic and amphiphilic surfactants.

2. (Cancelled).

3. The composition of claim 1, wherein said human keratin fibers are hair.

4. The composition of claim 1, wherein said at least one branched sulphonic polyester is formed by polymerizing:
at least one difunctional dicarboxylic acid having no sulphonic functional groups;
at least one difunctional monomer having at least one sulphonic functional group, wherein said at least one functional group is chosen from hydroxyl, carboxyl and amino groups;
at least one diol or a mixture of at least one diol and at least one diamine; and
at least one multifunctional reagent having at least three functional groups chosen from amino, alcohol and carboxylic acid groups.

5. The composition of claim 4, wherein the mixture polymerized further includes
at least one difunctional monomer chosen from hydroxycarboxylic acids and aminocarboxylic acids.

6. The composition of claim 5, wherein said polymerization utilizes
2 to 15 mol % of said at least one difunctional monomer having at least one sulphonic function;
0 to 40 mol % of said at least one difunctional monomer chosen from hydroxycarboxylic acids and aminocarboxylic acids; and
0.1 to 40 mol % of said at least one multifunctional reagent bearing at least three reactive functional groups.

7. The composition of claim 4, wherein said polymer contains substantially equal proportions, in terms of number of equivalents, of carboxylic acid groups and of diol or diol and diamine groups.

8. The composition of claim 4, wherein said at least one difunctional dicarboxylic acid having no sulphonic functional groups is chosen from aliphatic dicarboxylic acids, alicyclic dicarboxylic acids, and aromatic dicarboxylic acids.

9. The composition of claim 8, wherein said at least one difunctional dicarboxylic acid having no sulphonic functional groups is chosen from 1,4-cyclohexanedioic acid, succinic acid, glutaric acid, adipic acid, azelaic acid, sebacic acid, fumaric acid, maleic acid, 1,3-cyclohexanedioic acid, phthalic acid, terephthalic acid, and isophthalic acid.

10. The composition of claim 4, wherein said at least one difunctional monomer having at least one sulphonic functional group is chosen from dicarboxylic acids, dicarboxylic acid esters, glycols and hydroxy acids each containing at least one metal sulphonate group.

11. The composition of claim 4, wherein said at least one diol is chosen from alkanediols and polyalkylenediols.

12. The composition of claim 11, wherein said at least one diol is chosen from ethylene glycol, propylene glycol, diethylene glycol, triethylene glycol and polypropylene glycol.

13. The composition of claim 4, wherein said at least one diamine is chosen from alkanediamines and polyalkylenediamines.

14. The composition of claim 4, wherein said at least one multifunctional reagent is chosen from trimethylolethane, trimethylolpropane, glycerol, pentaerythritol, sorbitol, trimellitic anhydride, erythritol, threitol, dipentaerythritol, pyromellitic dianhydride and dimethylpropionic acid.

15. The composition of claim 1, wherein said at least one branched sulphonic polyester is present in amount ranging from 0.01 to 40% by weight relative to the total weight of the composition.

16. The composition of claim 15, wherein said at least one branched sulphonic polyester is present in amount ranging from 0.1 to 16% by weight relative to the total weight of the composition.

17. The composition of claim 16, wherein said at least one branched sulphonic polyester is present in amount ranging from 0.5 to 4% by weight relative to the total weight of the composition.

18. The composition of claim 1, wherein said at least one branched sulphonic polyester is in dissolved form or in the form of a dispersion of solid particles.

19. The composition of claim 1, wherein said at least one conditioning agent is insoluble and is chosen from poly-α-olefins, fluoro oils, plant oils, natural waxes, fluoro waxes, fluoro gums, fatty acid esters, insoluble silicones and amide compounds comprising at least one fatty chain.

20. The composition of claim 1, wherein said cationic and amphoteric polymers are chosen from
cationic cellulose derivatives,
dimethyldiallylammonium halide homopolymers,
dimethyldiallylammonium halide copolymers,
polyquaternary ammonium polymers,
vinylpyrrolidone copolymers containing cationic units, and
cationic polysiloxanes.

21. The composition of claim 1, wherein said cationic polymers are chosen from
quaternary cellulose ether derivatives,
copolymers of cellulose with a water-soluble quaternary ammonium monomer,
cyclopolymers,
cationic polysaccharides,
cationic silicone polymers,
quaternized and non-quaternized vinylpyrrolidone/dialkylaminoalkyl acrylate and methacrylate copolymers,
quaternary polymers of vinyl pyrrolidone and of vinylimidazole, and polyamidoamines.

22. The composition of claim 1, wherein said cationic surfactants are water-insoluble and are chosen from fatty amines and salts thereof.

23. The composition of claim 1, wherein said cationic surfactants are water-insoluble and are chosen from quaternary ammonium salts.

24. The composition of claim 22, wherein said fatty amines are
chosen from dioctylamine,
stearyidimethylamine,
palmityidimethylamine,
oleocetydimethylamine and
amidoamines.

25. The composition of claim 24, wherein said amidoamines are chosen from stearylamidoethyldiethylamine, behenylamidopropyldimethylamine, stearylamidopropyl-dimethylamine, oleylamidopropyldimethylamine and stearylamidoethyldimethylamine.

26. The composition of claim 23, wherein said cationic surfactants have the following formula (I), 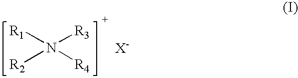
in which R₁ to R₄, which may be identical or different, are chosen from aliphatic radicals comprising from 1 to 22 carbon atoms and aromatic, alkoxy, polyoxyalkylene, alkylamide, hydroxyalkyl, aryl and alkylaryl radicals comprising from 12 to 22 carbon atoms; and X is an anion chosen from halides, phosphates, acetates, lactates and alkylsulphates.

27. The composition of claim 23, wherein said cationic surfactants comprise at least two quaternary imidazolinium salts of formula (II) below: 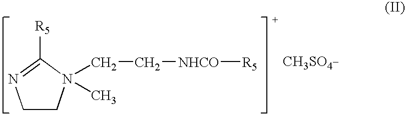
in which R. is chosen from alkenyl and alkyl radicals comprising from 13 to 21 carbon toms and fatty acid derivatives of tallow,

28. The composition of claim 23, wherein said cationic surfactants have the formula (III): 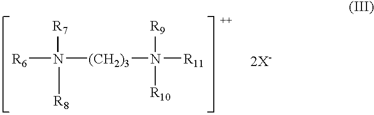
in which R₆ is chosen from aliphatic radicals comprising from 16 to 22 carbon atoms, R₇, R₈, R₉, R₁₀ and R₁₁ are chosen from hydrogen and alkyl radicals comprising from 4 carbon atoms, and X is an anion chosen from halides, acetates, phosphates and sulphates.

29. The composition of claim 1, wherein said composition further comprises at least one cosmetic additive.

30. The composition of claim 29, wherein said at least one cosmetic additive is chosen from plasticizers and neutralizing agents.

31. The composition of claim 1, wherein said composition is in the form of a vaporizable composition, a mousse, a gel or a lotion.

32. The composition of claim 1, wherein said composition further comprises a solvent chosen from water, an alcohol, and an aqueous-alcoholic mixture.

33. The composition of claim 29, wherein the composition further comprises a suitable amount of at least one propellant.

34. The composition of claim 33, wherein said at least one propellant is chosen from compressed gas and liquefied gases.

35. The composition of claim 33, wherein said at least one propellant is chosen from air, carbon dioxide and nitrogen.

36. The composition of claim 33, wherein said at least one propellant is chosen from gas which is soluble or insoluble in the composition.

37. The composition of claim 36, wherein said propellant is chosen from dimethyl ether, fluoro and non-fluoro hydrocarbons.

38. An aerosol device comprising a container containing in a liquid phase an aerosol composition a cosmetic composition in a suitable solvent, a propellant, and a means for dispensing said aerosol composition, wherein said cosmetic composition comprises:
at least one branched sulphonic polyester; and
at least one conditioning agent chosen from non-volatile silicones, cationic and amphoteric polymers and cationic and amphiphilic surfactants.

39. The aerosol device of claim 38, wherein said at least one branched sulphonic polyester is formed by polymerizing;
at least one difunctional dicarboxylic acid having no sulphonic functional groups;
at least one difunctional monomer having at least one sulphonic functional group, wherein said at least one functional group is chosen from hydroxyl, carboxyl and amino groups;
at least one diol or a mixture or at least one diol and at least one diamine; and
at least one multifunctional reagent having at least three functional groups chosen from amino, alcohol and carboxylic acid groups.

40. The aerosol device of claim 39, wherein the mixture polymerized further includes
at least one difuntional monomer chosen from hydroxycarboxylic acids and aminocarboxylic acids.

41. A process for treating keratin fibers comprising applying a cosmetic composition to the fibers, either before or after shaping the hairstyle, wherein said cosmetic composition comprises:
at least one branched sulphonic polyester; and
at least one conditioning agent chosen from non-volatile silicones, cationic and amphoteric polymers and cationic and amphiphilic surfactants.

42. The process of claim 41, wherein said at least one branched sulphonic polyester is formed by polymerizing:
at least one difunctional dicarboxylic acid having no sulphonic functional groups;
at least one difunctional monomer having at least one sulphonic functional group, wherein said at least one functional group is chosen from hydroxyl, carboxyl and amino groups;
at least one diol or a mixture of at least one diol and at least one diamine; and
at least one multifunctional reagent having at least three functional groups chosen from amino, alcohol and carboxylic acid groups.

43. The process of claim 42, wherein the mixture polymerized further includes
at least one difunctional monomer chosen from hydroxycarboxylic acids and aminocarboxylic acids.

44. A method of making a cosmetic styling formulation comprising the step of including in said formulation
at least one branched sulphonic polyester; and
at least one conditioning agent chosen from non-volatile silicones, cationic and amphoteric polymers and cationic and amphiphilic surfactants.
